(19) United States
(12) Patent Application Publication
(10) Pub. No.: US 2025/0268497 A1
(43) Pub. Date: Aug. 28, 2025

(54) CLOSED SYSTEM BLOOD TRANSFER DEVICE

(71) Applicant: LifeOutcomes LLC, Englewood, CO (US)

(72) Inventors: Robert J. McKINNON, Highlands Ranch, CO (US); Dean H. IWASAKI, Denver, CO (US)

(73) Assignee: LifeOutcomes LLC, Englewood, CO (US)

(21) Appl. No.: 18/858,249

(22) PCT Filed: Apr. 25, 2023
(86) PCT No.: PCT/US23/66154

§ 371 Date: Oct. 18, 2024

Related U.S. Application Data
(60) Provisional application No. 63/363,583, filed on Apr. 26, 2022.
(60) Provisional application No. 63/406,109, filed on Sep. 13, 2022.
(60) Provisional application No. 63/430,940, filed on Dec. 7, 2022.

(51) Int. Cl. A61B 5/15

(52) U.S. Cl. CPC A61B 5/150992; A61B 5/15003; A61B 5/150221; A61B 5/150786

(57) ABSTRACT

A blood sequestration system is provided that does not employ a mechanical means for selectively directing waste blood to a sample collection bottle, such as a blood culture bottle. The system employs a syringe to collect waste blood, wherein the syringe creates a negative pressure that actuates a one-way valve that prevents collected blood from being directed toward the sample collection bottle. After a predetermined amount of waste blood is collected, the sample collection bottle is selectively opened to receive sample blood, wherein the waste blood is prevented from tainting the sample blood.

This application is a national stage application under 35 U.S.C. 371 and claims the benefit of PCT Application No. PCT/US23/66154 having an international filing date of Apr. 25, 2023, which designated the United States, which claims the benefit of U.S. Provisional Patent Application Ser. No. 63/363,583, filed Apr. 26, 2023, U.S. Provisional Patent Application Ser. No. 63/406,109, filed Sep. 13, 2022, and U.S. Provisional Patent Application Ser. No. 63/430,940, filed Dec. 7, 2022, the disclosure of each of which are incorporated by reference herein.

### FIELD OF THE INVENTION

Embodiments of the present invention are related to preventing or reducing false-positive blood culture tests. Other embodiments of the present invention are related to devices that ensure blood collected in a vial or bottle is sterile.

### BACKGROUND OF THE INVENTION

Collecting and testing blood cultures from patients to determine blood infections like sepsis is common. However, approximately 40% of positive blood culture tests indicating the presence of a pathogen are false positive due to blood contamination occurring during collection, which means approximately 1.2 million U.S. patients annually are affected by false-positive blood cultures. To address this issue, an initial amount of “waste blood” is commonly drawn from a patient and sequestered. A predetermined volume of “sample blood” is then directed to a blood culture bottle or tube. The blood culture bottle or tube may include a medium that promotes the growth of blood-borne pathogens. As one of ordinary skill in the art will appreciate, it is desirous to ensure post-collection tests accurately reflect the presence of pathogens in the patient's blood, not on their dermis or in the blood collection system.

Common blood collection devices employ blood diversion systems that sequester waste blood and then manually transitions blood flow to a collection bottle or tube. Such systems utilize selectively movable valves, stopcocks, or other complicated blood diversion mechanisms that are difficult to use and expensive to manufacture. One of ordinary skill in the art will appreciate that ease of use is directly proportional to human error, which can also lead to false positive test results.

Accordingly, embodiments of the present invention described herein are directed to easy-to-use systems for collecting an initial amount of blood from a patient, sequestering the initial amount of blood, and collecting a blood sample for testing, wherein blood diversion from the initial collection volume to the sample collection is simplified.

### SUMMARY OF THE INVENTION

It is one aspect of some embodiments of the present invention to provide a closed blood transfer device, which reduces the chances of contamination. Further, the contemplated system is operator-friendly and intuitive, which minimizes the chance of errors and blood loss.

It is another aspect of some embodiments of the present invention to provide a system generally comprising an infusion set terminating in a needle configured for drawing blood from a patient. The needle is interconnected to tubing that leads to a fluid diverter, such as a T or Y connector. The connector includes an outlet in fluidic communication with a unidirectional valve that allows fluid to pass in one direction toward an associated syringe. The unidirectional valve may be a common in-line check valve that employs a diaphragm. In other embodiments, the valve consists of a hydrophilic material that allows fluid to be transferred therethrough for a predetermined time before it seals, thereby shutting off all fluid flow. Hydrophilic flow control devices are described in U.S. Patent Application Publication No. 2008/0312576 and U.S. Pat. No. 7,090,646, which are incorporated by reference herein.

Another connector outlet is interconnected to a tube holder configured to receive a blood culture collection vial, tube, syringe, or bag. One of ordinary skill in the art will appreciate that the tube holder may be specifically designed to accept a certain type of blood collection means. In other embodiments, a universal tube holder designed to accept different types of blood collection means. The tube holder includes a needle designed to puncture the blood culture collection means. In one embodiment, the needle is surrounded by a sheath, thereby closing the system before the needle pierces the collection means, which provides additional system internal volume to receive sample blood.

As alluded to above, the sample collection means may be a capped syringe. In operation, the syringe is inserted into the bottle holder or similar device, where the needle shroud is eventually ruptured. Further syringe insertion will cause the needle to pierce the elastomeric cap so that blood can enter the syringe barrel when the plunger is retracted. The syringe cap will effectively seal the syringe when it is removed from the bottle holder. In other embodiments, the contemplated sample syringe connects to the sample outlet of the connector with a luer connector, for example. Such a syringe may employ a post-collection sealing device, e.g., the aforementioned hydrophilic filter, to prevent leakage after the sample is drawn, or a cap/stopper is placed over the syringe end after it is removed from the connector.

One of ordinary skill in the art will appreciate that the components briefly mentioned above may be selectively interconnected by common luer locks, threads, bayonet fittings, or utilize interference fits. However, some embodiments of the present invention employ systems wherein one or more component interconnections are ultrasonically welded, snap fit, or glued. Regardless of interconnection type or types, the systems of embodiments of the present invention are substantially leak-free.

The blood sequestration system of one embodiment directs blood taken from the patient to the connector, having the blood inlet, the first blood outlet associated with “waste” blood, and the second blood outlet associated with “sample” blood to be tested. The first outlet is in fluid communication with the unidirectional valve that opens when exposed to upstream negative pressure generated when the syringe plunger is drawn outwardly from the syringe body. As one of ordinary skill in the art will appreciate, plunger release or the application of positive pressure with the syringe (i.e., pushing the plunger into the syringe body) closes the unidirectional valve, which prevents additional blood from bypassing the valve and entering the syringe. The syringe may include a marking indicating a minimum draw volume, for example, 2 milliliters. In one embodiment, the syringe barrel is substantially clear or devoid of visual obstructions to increase user awareness of the amount of blood collected. The second outlet of the connector is in fluid communication with the blood culture collection tube, syringe, blood bag, tube, or any other fluid collection means.

In operation, a user inserts a needle into the patient's vein or interconnects tubing to an extension set configured for interconnection to an intravenous (IV) line. Blood urged by the patient's blood pressure and/or negative pressure generated by the syringe is directed to the connector's inlet. Blood flows through the unidirectional valve, opened by negative pressure generated when the user pulls the syringe plunger. Eventually, a predetermined amount of blood fills the syringe, wherein the unidirectional valve prevents waste blood from re-entering the connector. After a predetermined amount of waste blood has been captured in the syringe, at least one blood culture collection bottle or tube is inserted into the tube holder, breaking the protective needle shroud. Because negative pressure generation by the syringe has ceased, blood flows from the patient into the sample blood collection means. One of ordinary skill in the art will appreciate that the sample blood collection means may possess a negative pressure, which facilitates blood flow towards the same. Those of ordinary skill in the art will also appreciate that a secondary valve, e.g., a rotatable stopcock or a slidable flow control device as described in U.S. Pat. No. 10,328,212, which is incorporated by reference herein, may be provided that effectively closes the connector's second outlet to waste blood flow.

One of ordinary skill in the art will appreciate that instead of a syringe for collecting waste blood, a vacuum bottle or tube may be employed that may be selectively interconnected to a tube holder interconnected to the connector's first outlet, wherein negative pressure in the tube/bottle draws waste blood through the unidirectional valve. Internal system pressure eventually equalizes when the tube/bottle is filled to a predetermined degree, which may close the valve, preventing further sample blood collection. Of course, if the tube/bottle is full, additional blood will be prevented from flowing out of the connector's first outlet. In addition, the contemplated tube holder and blood culture collection tube/bottle described above may be replaced with a larger syringe.

In some embodiments, air pressure residing in the small annulus defined by an internal volume between the connector's second outlet, the tube holder's interface with the connector, and the internal volume of the needle, which is effectively capped by the shroud, creates an air barrier (e.g., a bubble). The air barrier prevents waste blood from flowing substantially toward the second outlet, especially when the syringe is generating negative pressure. Once the needle's shroud is ruptured, negative pressure associated with the collection bottle or tube effectively forces the air bubble into the collection bottle or tube, which has been found not to substantially introduce a sufficient amount of contaminants or pathogens into the sample blood. That is, the amount of air found in the small annulus is insufficient to promote the growth of pathogens or adversely effect anaerobic blood culture tests.

It is an aspect of some embodiments of the present invention to provide blood collection vials, tubes, bottles, etc., that are easy to sterilize before use. More specifically, sample blood is often directed into the aforementioned negatively pressurized blood collection means, e.g., a tube. Such blood collection tubes are sealed with a cap that must be sterilized before it is punctured by a needle to ensure the collected sample remains contamination-free. Unfortunately, many existing blood collection tubes have caps that are difficult or impossible to clean effectively. Accordingly, some embodiments of the present invention employ a tube with a generally flat stopper or a stopper protected by a flat cover seal that is easier to clean and disinfect. Accordingly, secondary tube-specific packaging is generally not required. The interior annulus of the vial may be sterilized using known radiation methods. The contemplated tubes may be placed in a sterilized single-tube blister pack to further increase sterility. Alternatively, a plurality of tubes with cover seals may be provided in a tray.

The contemplated cover may also confirm that the vial has been wiped. More specifically, a disinfection indicator may be provided on the cover seal. For example, water-soluble or alcohol-soluble indicia may be provided that allows the user to verify when the cover has been wiped. For example, some cover seals possess a red dot that disappears when the seal is wiped. Other cover seals react with disinfecting solutions and display a message, such as “clean” or “OK,” for a predetermined period after exposure to disinfecting solution. Alternatively, the cover will change colors when exposed to air, wiped with alcohol or another fluid, etc.

Thus, it is one aspect of some embodiments of the present invention to provide a system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; and wherein the second needle is adapted to rupture the shroud and puncture the means for blood collection when the means for blood collection is inserted into the tube holder.

It is one aspect of other embodiments of the present invention to provide a system for sequestering blood and providing a blood sample comprising: a primary connector having an inlet, a first outlet, and a second outlet; a tube adapted to receive blood the tube having an end that is associated with the inlet; a valve associated on one end with the first outlet, and on a second end with a waste blood collection vessel; and a tube holder having a first end configured to interconnect to the second outlet and a second, open end adapted to receive a sample collection vessel, the tube holder further comprising a needle extending from the first end toward the second, open end, wherein the needle is surrounded by a selectively rupturable shroud.

Further aspects of the present invention are provided in the following embodiments:

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and wherein blood drawn from the patient is prevented from substantially flowing through the second outlet until the blood collection means is inserted into the tube holder.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; wherein blood drawn from the patient is prevented from substantially flowing through the second outlet until the blood collection means is inserted into the tube holder; and wherein an air bubble associated with the second outlet prevents a substantial amount of blood from exiting the second outlet.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and wherein blood drawn from the patient is prevented from substantially flowing into the second needle until the blood collection means is inserted into the tube holder.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and wherein the connector is not associated with a mechanical device for diverting blood flow from the first outlet to the second outlet.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and wherein the valve is permanently interconnected on one side to the primary connector and selectively interconnected on the other end to the syringe.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and wherein the connector associated with the tube is a first connector, and further comprising a second connector positioned between the second outlet and the valve, and a third connector positioned between the valve and the syringe.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and wherein the tube holder is configured to receive a means for blood collection of various sizes.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and the means for blood collection is a tube or blood culture bottle.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and wherein the means for blood collection is a tube having an open end, a cap having a puncturable seal that seals the open end, and a cover having a substantially flat outer surface interconnected to an upper surface of the cap.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and the means for blood collection is a tube having an open end, a cap having a puncturable seal that seals the open end, and a cover having a substantially flat outer surface interconnected to an upper surface of the cap; and wherein the outer surface includes indicia that is removed or altered when exposed to water, alcohol, or other liquid.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and the means for blood collection is a tube having an open end, a cap having a puncturable seal that seals the open end, and a cover having a substantially flat outer surface interconnected to an upper surface of the cap; and wherein the outer surface includes indicia that is removed or altered when exposed to air.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and the means for blood collection is a tube having an open end, a cap having a puncturable seal that seals the open end, and a cover having a substantially flat outer surface interconnected to an upper surface of the cap; and wherein the outer surface includes indicia that changes when exposed to water, alcohol, or other liquid.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and the means for blood collection is a tube having an open end, a cap having a puncturable seal that seals the open end, and a cover having a substantially flat outer surface interconnected to an upper surface of the cap; and wherein the outer surface includes indicia that changes when exposed to air.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and the means for blood collection is a tube having an open end, a cap having a puncturable seal that seals the open end, and a cover having a substantially flat outer surface interconnected to an upper surface of the cap; and wherein the outer surface changes color when exposed to water, alcohol, or other liquid.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and the means for blood collection is a tube having an open end, a cap having a puncturable seal that seals the open end, and a cover having a substantially flat outer surface interconnected to an upper surface of the cap; and wherein the outer surface changes color when exposed to air.

A system for sequestering blood and providing a blood sample comprising: a primary connector having an inlet, a first outlet, and a second outlet; a first needle configured for drawing blood from a patient; a tube extending from the first needle and ending in a connector configured to interconnect to the inlet; a unidirectional valve associated on one end with the first outlet, and on a second end with a syringe; a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud; wherein the first needle is adapted to draw blood from a patient that is directed to the primary connector and out of the first outlet, and wherein blood is drawn into the syringe through the valve when a plunger of the syringe is pulled to create a negative pressure; wherein the second needle is adapted to rupture the shroud and puncture the means for blood collection when the means for blood collection is inserted into the tube holder; and the means for blood collection is a tube having an open end, a cap having a puncturable seal that seals the open end, and a cover having a substantially flat outer surface interconnected to an upper surface of the cap; and wherein the outer surface changes color when exposed to air, the outer surface further comprising indicia that removed or altered when exposed to water, alcohol, or other liquid. A system for sequestering blood and providing a blood sample comprising: a primary connector having an inlet, a first outlet, and a second outlet; a tube adapted to receive blood the tube having an end that is associated with the inlet; a valve associated on one end with the first outlet, and on a second end with a waste blood collection vessel; a tube holder having a first end configured to interconnect to the second outlet and a second, open end adapted to receive a sample collection vessel, the tube holder further comprising a needle extending from the first end toward the second, open end, wherein the needle is surrounded by a selectively rupturable shroud; wherein blood drawn from a patient that is directed to the primary connector and out of the first outlet, and wherein blood is drawn into the blood collection vessel; and wherein the needle is adapted to rupture the shroud and puncture the sample collection vessel is inserted into the tube holder.

A system for sequestering blood and providing a blood sample comprising: a primary connector having an inlet, a first outlet, and a second outlet; a tube adapted to receive blood the tube having an end that is associated with the inlet; a valve associated on one end with the first outlet, and on a second end with a waste blood collection vessel; a tube holder having a first end configured to interconnect to the second outlet and a second, open end adapted to receive a sample collection vessel, the tube holder further comprising a needle extending from the first end toward the second, open end, wherein the needle is surrounded by a selectively rupturable shroud; wherein blood drawn from a patient that is directed to the primary connector and out of the first outlet, and wherein blood is drawn into the blood collection vessel; wherein the needle is adapted to rupture the shroud and puncture the sample collection vessel is inserted into the tube holder; and wherein the blood collection vessel is a syringe, and the valve is a one-way valve that is opened when negative pressure is produced by the syringe.

A system for sequestering blood and providing a blood sample comprising: a primary connector having an inlet, a first outlet, and a second outlet; a tube adapted to receive blood the tube having an end that is associated with the inlet; a valve associated on one end with the first outlet, and on a second end with a waste blood collection vessel; a tube holder having a first end configured to interconnect to the second outlet and a second, open end adapted to receive a sample collection vessel, the tube holder further comprising a needle extending from the first end toward the second, open end, wherein the needle is surrounded by a selectively rupturable shroud; wherein blood drawn from a patient that is directed to the primary connector and out of the first outlet, and wherein blood is drawn into the blood collection vessel; wherein the needle is adapted to rupture the shroud and puncture the sample collection vessel is inserted into the tube holder; and wherein the system is devoid of a device for selectively altering fluid flow.

A system for sequestering blood and providing a blood sample comprising: a primary connector having an inlet, a first outlet, and a second outlet; a tube adapted to receive blood the tube having an end that is associated with the inlet; a valve associated on one end with the first outlet, and on a second end with a waste blood collection vessel; a tube holder having a first end configured to interconnect to the second outlet and a second, open end adapted to receive a sample collection vessel, the tube holder further comprising a needle extending from the first end toward the second, open end, wherein the needle is surrounded by a selectively rupturable shroud; and wherein blood drawn from the patient is prevented from substantially flowing through the second outlet until the sample collection vessel is inserted into the tube holder.

The Summary of the Invention is neither intended nor should it be construed as being representative of the full extent and scope of the present invention. That is, these and other aspects and advantages will be apparent from the disclosure of the invention(s) described herein. Further, the above-described embodiments, aspects, objectives, and configurations are neither complete nor exhaustive. As will be appreciated, other embodiments of the invention are possible using, alone or in combination, one or more of the features set forth above or described below. Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention as well as in the attached drawings and the Detailed Description and no limitation as to the scope of the present invention is intended by either the inclusion or non-inclusion of elements, components, etc. in this Summary of the Invention. Additional aspects of the present invention will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present invention are possible utilizing, alone or in combination, as set forth above and/or described in the accompanying figures and/or in the description herein below.

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and drawing figures are to be understood as being approximations which may be modified in all instances as required for a particular application of the novel assembly and method described herein.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C., Section 112 (f). Accordingly, a claim incorporating the term “means” shall cover all structures, materials, or acts set forth herein, and all of the equivalents thereof.

### BRIEF DESCRIPTION OF THE DRAWINGS

The accompanying drawings, which are incorporated in and constitute a part of the specification, illustrate embodiments of the invention and, together with the general description of the invention given above and the detailed description of the drawings given below, serve to explain the principles of these inventions.

FIG. 1 is a blood sequestration system of one embodiment of the present invention.

FIG. 2 is an exploded view of the blood sequestration system shown in FIG. 1.

FIG. 3 is a detailed view of FIG. 1.

FIG. 4 is a blood collection tube of one embodiment of the present invention.

The following component list and associated numbering found in the drawings is provided to assist in the understanding of one embodiment of the present invention:

| # | Component |
|---|---|
| 2 | System |
| 6 | Winged infusion set |
| 10 | Tube holder |
| 14 | Syringe |
| 18 | Connector |
| 22 | Tubing |
| 26 | Needle |
| 30 | Connector |
| 34 | Needle |
| 38 | Shroud |
| 42 | Tube/bottle |
| 46 | Cover |
| 50 | Housing |
| 54 | Plunger |
| 58 | Indicia |
| 62 | Inlet |
| 66 | Waste blood outlet |
| 70 | Sample blood outlet |
| 74 | Connector |
| 78 | Male-to-male luer connector |
| 82 | First end |
| 86 | Valve |
| 90 | Female-to-female luer connector |
| 94 | Syringe opening |
| 100 | Air bubble |
| 104 | Blood |
| 142 | Tube |
| 150 | Cap |
| 154 | Seal |
| 158 | Indentation |
| 162 | Cover |

It should be understood that the drawings are not necessarily to scale. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION

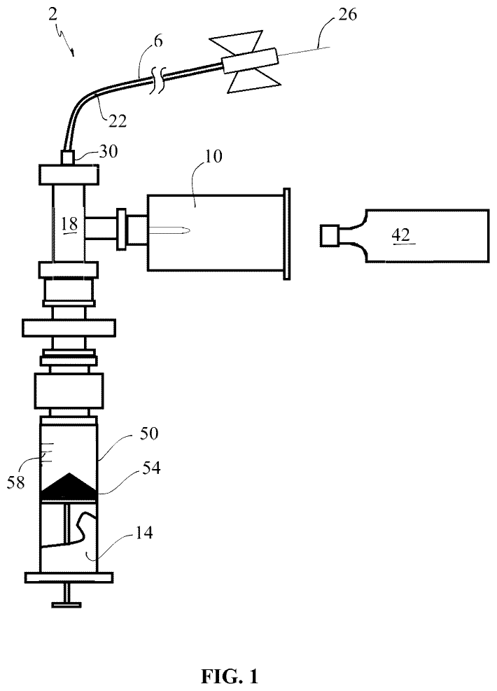

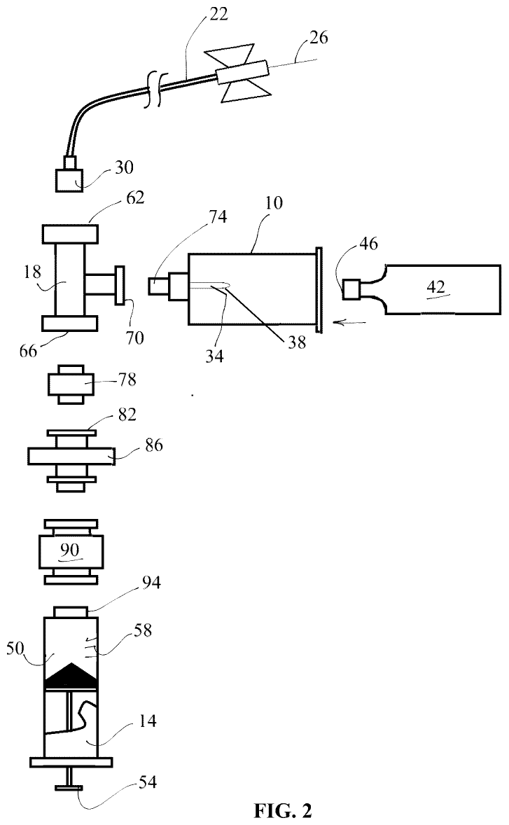

FIGS. 1-3 show the blood sequestration system 2 of one embodiment of the present invention that generally comprises a winged infusion set 6, a tube holder 10, and a syringe 14 that are in fluid communication with a connector 18. The winged infusion set 6 further consists of tubing 22 with a needle 26 at one end and a connector 30 at the other. One of ordinary skill in the art will appreciate that a needleless set can be used to sample from a venous catheter. In one embodiment of the present invention, the tube is about 12 inches long. The infusion set's connector 30 may be a common luer connector.

The tube holder 10 accommodates a needle 34 covered before use with a shroud 38. The tube holder 10 is sized to receive a blood collection bottle, vial, or tube 42 (hereinafter, “tube”), which has a cover 46 that is pierced by the needle 34 when the tube 42 is inserted into the tube holder 10. The tube may possess negative pressure, such that when the cover 46 is pierced, blood is drawn into the tube 42.

The syringe 14, which should be well understood by those of skill in the art, comprises a housing 50 that accommodates a plunger 54. The syringe may also include a plurality of indicia 58 that shows the amount of waste blood drawn into the syringe.

The connector 18 may be a T or Y connector that includes a fluid inlet 62, a waste blood outlet 66, and a sample blood outlet 70. As mentioned above, the connector may include a manual valve that shifts blood flow from the waste blood outlet to the sample blood outlet.

The above-mentioned components are interconnected in one embodiment of the present invention using a plurality of connectors. For example, the connector 30 of the wing set is interconnected to a corresponding interface provided by the connector 18. The blood sample outlet may also have a luer lock port for receipt of a corresponding connector 74 provided on the tube holder 10. The waste blood outlet may also be configured to receive one end of a male-to-male luer connector 78, wherein the other end of the connector 78 is interconnected to a first end 82 of a unidirectional valve 86. The other end of the unidirectional valve 86 is interconnected to a female-to-female luer connector 90. Finally, a syringe opening 94 is interconnected to the female-to-female luer connector 94, thereby providing a closed system. One of ordinary skill in the art will appreciate that one or more of the interconnection points may be permanently or semi-permanently interconnected or fused, e.g., by ultrasonically welding, to define various subassemblies. For example, the connector and valve of one embodiment are comprised of a single unit. Accordingly, various connectors mentioned herein may be omitted so long as the connector 18, tube holder 10, valve 86, and syringe 14 are in fluidic communication.

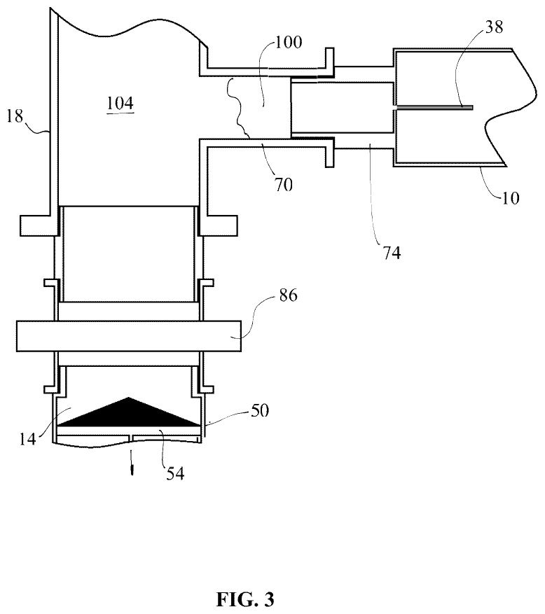

FIG. 3 is a cross-section showing a portion of the system of one embodiment of the present invention. Here, one of ordinary skill in the art will appreciate that before the shroud is pierced, a secondary “valve” comprising an air bubble 100 may be provided. More specifically, when blood 104 initially enters the system and encounters the connector 18, air initially in the system having a first pressure (e.g., atmospheric pressure) propagates towards the patient and eventually equalizes with the patient's blood pressure. However, it is conceivable that air between the sample blood outlet 70 and the bottle holder (i.e., the air bubble described above) will be trapped and compressed by the pressure associated with the blood in the connector 18. As the plunger 54 is retracted, blood passes through the unidirectional valve 86 and into the syringe 14. Because the negative pressure generated by the syringe is less than that of the blood pressure in the system, blood flows into the syringe and not out of the sample blood outlet 70.

The small air bubble 100 prevents a substantial amount if not all, waste blood from reaching the blood sample outlet 70. It is conceivable, yet highly unlikely (and probably impossible, because of syringe volume, generated negative pressures, and normal human blood pressures), that the negative pressure generated by the syringe will pull the air bubble into the connector 18. If so, the pressure associated with the air bubble would equalize with that of the blood pressure provided in the system, or the air bubble will be drawn into the syringe. Again, the amount of air provided by the air bubble 100 is minimal but sufficient to prevent blood from entering the tube holder's needle under normal conditions.

After a predetermined amount of waste blood is collected within the syringe, the plunger is released, which prevents further fluid flow through the valve 86. The tube 42 is then inserted into the tube holder 10, wherein the needle 34 eventually punctures the cover 46 of the tube 42, allowing blood 104 to flow through the sample blood outlet 70, through the needle 34, into the collection tube 42. Accordingly, blood is sequestered in the syringe using only the valve 86 and syringe 14. More specifically, the blood diversion mechanism of one embodiment of the present invention is devoid of any mechanical fluid diversion devices, such as a manually or automatically actuated valve.

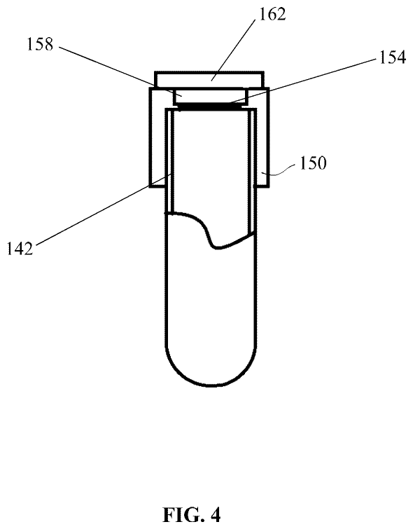

FIG. 4 shows a blood collection tube 142 of one embodiment of the present invention. The tube includes a cap 150 that includes a seal 154 provided in an indentation 158. As one of ordinary skill in the art will appreciate, maintaining the cleanliness of the tube seal 154 in light of the indentation 58 is extremely difficult. Accordingly, one embodiment of the present invention employs a cover 162 over the indentation 158. In operation, the cover 162 is placed over the tube seal 154 after it is initially factory cleaned and disinfected. Thereafter, the sealed tube can be irradiated to sterilize the tube's inner volume and air annulus between the cover 162 and the tube seal 154.

Exemplary characteristics of embodiments of the present invention have been described. However, to avoid unnecessarily obscuring embodiments of the present invention, the preceding description may omit several known apparatus, methods, systems, structures, and/or devices one of ordinary skill in the art would understand are commonly included with the embodiments of the present invention. Such omissions are not to be construed as a limitation of the scope of the claimed invention. Specific details are set forth to provide an understanding of some embodiments of the present invention. It should, however, be appreciated that embodiments of the present invention may be practiced in a variety of ways beyond the specific detail set forth herein.

Modifications and alterations of the various embodiments of the present invention described herein will occur to those skilled in the art. It is to be expressly understood that such modifications and alterations are within the scope and spirit of the present invention, as set forth in the following claims. Further, it is to be understood that the invention(s) described herein is not limited in its application to the details of construction and the arrangement of components set forth in the preceding description or illustrated in the drawings. That is, the embodiments of the invention described herein are capable of being practiced or of being carried out in various ways. The scope of the various embodiments described herein is indicated by the following claims rather than by the foregoing description. And all changes which come within the meaning and range of equivalency of the claims are to be embraced within their scope. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The foregoing disclosure is not intended to limit the invention to the form or forms disclosed herein. In the foregoing Detailed Description, for example, various features of the invention are grouped together in one or more embodiments for the purpose of streamlining the disclosure. This method of disclosure is not to be interpreted as reflecting an intention that the claimed inventions require more features than expressly recited. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention. Further, the embodiments of the present invention described herein include components, methods, processes, systems, and/or apparatus substantially as depicted and described herein, including various sub-combinations and subsets thereof. Accordingly, one of skill in the art will appreciate that would be possible to provide for some features of the embodiments of the present invention without providing others. Stated differently, any one or more of the aspects, features, elements, means, or embodiments as disclosed herein may be combined with any one or more other aspects, features, elements, means, or embodiments as disclosed herein.

What is claimed is:

1. A system for sequestering blood and providing a blood sample comprising:
a primary connector having an inlet, a first outlet, and a second outlet;
a first needle configured for drawing blood from a patient;
a tube extending from the first needle and ending in a connector configured to interconnect to the inlet;
a unidirectional valve associated on one end with the first outlet, and on a second end with a syringe;
a tube holder having a first end configured to interconnect to the second outlet and a second, open end adapted to receive a means for blood collection, the tube holder further comprising a second needle extending from the first end toward the second, open end, wherein the second needle is surrounded by a selectively rupturable shroud;
wherein the first needle is adapted to draw blood from a patient that is directed to the primary connector and out of the first outlet, and wherein blood is drawn into the syringe through the valve when a plunger of the syringe is pulled to create a negative pressure; and
wherein the second needle is adapted to rupture the shroud and puncture the means for blood collection when the means for blood collection is inserted into the tube holder.

2. The system of claim 1, wherein blood drawn from the patient is prevented from substantially flowing through the second outlet until the blood collection means is inserted into the tube holder.

3. The system of claim 2, wherein an air bubble associated with the second outlet prevents a substantial amount of blood from exiting the second outlet.

4. The system of claim 1, wherein blood drawn from the patient is prevented from substantially flowing into the second needle until the blood collection means is inserted into the tube holder.

5. The system of claim 1, wherein the connector is not associated with a mechanical device for diverting blood flow from the first outlet to the second outlet.

6. The system of claim 1, wherein the valve is permanently interconnected on one side to the primary connector and selectively interconnected on the other end to the syringe.

7. The system of claim 1, wherein the connector associated with the tube is a first connector, and further comprising a second connector positioned between the second outlet and the valve, and a third connector positioned between the valve and the syringe.

8. The system of claim 1, wherein the tube holder is configured to receive a means for blood collection of various sizes.

9. The system of claim 1, wherein the means for blood collection is a tube or blood culture bottle.

10. The system of claim 1, wherein the means for blood collection is a tube having an open end, a cap having a puncturable seal that seals the open end, and a cover having a substantially flat outer surface interconnected to an upper surface of the cap.

11. The system of claim 10, wherein the outer surface includes indicia that is removed or altered when exposed to water, alcohol, or other liquid.

12. The system of claim 10, wherein the outer surface includes indicia that is removed or altered when exposed to air.

13. The system of claim 10, wherein the outer surface includes indicia that changes when exposed to water, alcohol, or other liquid.

14. The system of claim 10, wherein the outer surface includes indicia that changes when exposed to air.

15. The system of claim 10, wherein the outer surface changes color when exposed to water, alcohol, or other liquid.

16. The system of claim 10, wherein the outer surface changes color when exposed to air.

17. The system of claim 10, wherein the outer surface changes color when exposed to air, the outer surface further comprising indicia that removed or altered when exposed to water, alcohol, or other liquid.

18. A system for sequestering blood and providing a blood sample comprising:
a primary connector having an inlet, a first outlet, and a second outlet;
a tube adapted to receive blood the tube having an end that is associated with the inlet;
a valve associated on one end with the first outlet, and on a second end with a waste blood collection vessel; and
a tube holder having a first end configured to interconnect to the second outlet and a second, open end adapted to receive a sample collection vessel, the tube holder further comprising a needle extending from the first end toward the second, open end, wherein the needle is surrounded by a selectively rupturable shroud.

19. The system of claim 18, wherein blood drawn from a patient that is directed to the primary connector and out of the first outlet, and wherein blood is drawn into the blood collection vessel; and
wherein the needle is adapted to rupture the shroud and puncture the sample collection vessel is inserted into the tube holder.

20. The system of claim 19, wherein the blood collection vessel is a syringe, and the valve is a one-way valve that is opened when negative pressure is produced by the syringe.

21. The system of claim 19, wherein the system is devoid of a device for selectively altering fluid flow.

22. The system of claim 18, wherein blood drawn from the patient is prevented from substantially flowing through the second outlet until the sample collection vessel is inserted into the tube holder.
